(19) United States
(12) Patent Application Publication
(10) Pub. No.: US 2018/0296233 A1
(43) Pub. Date: Oct. 18, 2018

(54) ASPIRATION CATHETER

(71) Applicant: STG MEDICAL AG, Winterthur (CH)

(72) Inventor: Michael SCHWAGER, Winterthur (CH)

(73) Assignee: STG MEDICAL AG, Winterthur (CH)

(21) Appl. No.: 15/771,810

(22) PCT Filed: Oct. 28, 2015
(86) PCT No.: PCT/CH2015/000157

§ 371 Date: Apr. 27, 2018

(51) Int. Cl. A61B 17/22

(52) U.S. Cl. CPC A61B 17/22; A61M 2039/229; A61M 2205/6081

(57) ABSTRACT

The invention relates to an aspiration catheter for the aspiration of material from blood vessels. The aspiration catheter according to the invention comprises a tubular catheter wall (2) having a first distal opening (3) and a first proximal opening (4). These two openings (3, 4) are connected to each other by a first lumen (5). The aspiration catheter further comprises a tubular guide sleeve (6) having a second distal opening (7) and a second proximal opening (8). A second lumen (9) extends between these two openings (7, 8). The tubular guide sleeve is arranged within the first lumen (5), wherein the second lumen (9) extends coaxially to the first lumen (5). The second proximal opening (8) extends laterally through the tubular catheter wall (2). The tubular guide sleeve is connected to the tubular catheter wall (2) in a region (X) of the first distal opening (3) and in a region (Y) of the second proximal opening (8). The invention further relates to a component kit for the aspiration of material from blood vessels and to a method for carrying out the same.

### TECHNICAL FIELD

The present invention relates to an aspiration catheter for the aspiration of material from blood vessels. The invention further relates to a component kit for the aspiration of material from blood vessels and to corresponding methods.

Further aspects of the invention concern the arrangement of radiopaque markers on aspiration catheters and also the color coding of component kits for the aspiration of material from blood vessels.

### PRIOR ART

Accumulations of endogenous or foreign material in a blood vessel may lead to partial or complete closure of the affected blood vessel. Such blockages may cause the supply of blood to tissues and organs to be impaired or completely cut off. Examples of such clinical pictures are infarcts, thromboses and embolisms. In the case of a thrombus, a plug of coagulated blood arises, while an embolus involves material in the blood vessels forming lumps, for example fats in the case of fat embolisms, or pus in the case of septic embolisms, which are also infected by bacteria.

Such blockages can occur in arteries and also veins. A particular form of arterial embolism can cause the infarction of the undersupplied tissue in question, which in turn can lead to a heart attack or stroke. In addition to other medical procedures that are used in acute cases and that serve for life support or emergency care of the patient, it is customary to remove the blockage material and/or to expand the affected vessel wall again. By means of catheters, the blockages are eliminated, i.e. suctioned off, and stents and/or balloons are then used to support or widen the affected tissue. Such interventions are performed percutaneously and are routine procedures, particularly in coronary interventions.

Specially adapted catheters are used for the aspiration of thrombi and/or emboli from blood vessels. In addition to having a diameter suitable for passage through the blood vessels, such catheters have an aspiration lumen through which, when a suction effect is applied at one end, aspiration from blood vessels can be performed through an aspiration opening provided at the other end. In addition to this first lumen, aspiration catheters also have at least one second lumen which serves to receive a guidewire. This guidewire is used to guide the catheter into the blood vessels and to place it at the intended aspiration site. A special type of catheter is a catheter of the rapid exchange type. In these catheters, the guidewire is guided only through a relatively short segment of the catheter. With such catheters, the operating surgeon can exchange the catheters more quickly and more easily. A guidewire lumen extends only in the guided region of the catheter, after which it emerges laterally from the catheter, and the guidewire in the vascular system runs substantially parallel and adjacent to the catheter.

For the suctioning of material, the catheter is advanced through the affected blood vessel as far as the obstructed site. Radiopaque markers on the catheter can permit precise localization and placement. When the catheter reaches the site of obstruction, it is pushed through the plug or embolus. The actual suctioning then takes place as the catheter is withdrawn, specifically from distal to proximal. As the aspiration opening is pulled through the material that is to be suctioned, said material is thus aspirated. To promote uniform aspiration, the catheter can also be rotated about its own longitudinal axis. Rotations of the catheter are occasionally also necessary during placement.

The catheter has to be advanced from the percutaneous opening to the site of obstruction. Decisive factors for the placement and safety of the catheter are its flexibility and also its stiffness. In conventional catheters, the at least one region in which the guidewire lumen is accommodated is responsible for a unilateral stiffening of the catheter. When the catheter is rotated, both during placement and also during the suction process, the catheter undergoes a flapping movement. That is to say, the rotation of the tip about its own axis is made difficult or impeded by preceding convexities of the catheter, specifically in such a way that uniform rotation is no longer possible, and instead a “flapping” of the catheter takes place. This not only reduces the effectiveness of the suction process, it also leads in addition to irritation of the inner wall of the vessel.

EP 1011775 B1 (Bagaoisan, C. J. et al.) describes an aspiration catheter which is suitable for suctioning platelet thrombi or other materials from vessels. The catheter shown consists of a long tubular body with a proximal end and a distal end. Between these ends there extends an aspiration lumen with an aspiration opening at the distal end. The catheter shown is a rapid exchange catheter, in which less than 40 cm of the distal region in the proximal direction has a guidewire lumen. Mounted at the proximal end is a syringe, which causes a suctioning effect by means of generation of a negative pressure. The guidewire lumen and the aspiration lumen are parallel and are completely separated. However, from the geometry of the catheter shown here, it is clear that this catheter is not suitable to be rotated about its own axis. The guidewire lumen extending externally along the aspiration lumen and parallel thereto impedes free rotation and causes flapping and twisting movements.

WO 2013/029795 A1 (Gulcher, M. et al.) also discloses an aspiration catheter of the rapid exchange type. This catheter is used to aspirate thrombotic material from blood vessels and has a first lumen and a second lumen. The first lumen forms the aspiration lumen, while the second lumen extends coaxially with respect to the first lumen and forms a guidewire lumen. This guidewire lumen, however, is secured to the catheter inner wall. This results in a unilateral stiffness of the catheter, and rotation about its own axis is correspondingly impeded.

There is therefore a need for an aspiration catheter which is easy to use and which overcomes at least one disadvantage of the known aspiration catheter.

### DISCLOSURE OF THE INVENTION

The object of the present invention is therefore to make available an aspiration catheter which overcomes at least one disadvantage of the known aspiration catheter. In particular, the aim is to make available an aspiration catheter, a component kit for the aspiration of material from blood vessels, and a method for its operation, which is simple and efficient to produce and preferably exhibits less flapping and/or twisting during rotation about its own axis.

This object has been achieved with an aspiration catheter, a component kit and/or a method according to the independent claims of the present invention.

One aspect of the present invention concerns an aspiration catheter for the aspiration of material from blood vessels. The aspiration catheter according to the invention comprises a tubular catheter wall having a first distal opening and a first proximal opening. These two openings are connected by a first lumen. The aspiration catheter additionally has a tubular guide sleeve having a second distal opening and a second proximal opening. The second distal opening and the second proximal opening are also connected by a second lumen. The tubular guide sleeve is arranged inside the first lumen. The second lumen extends coaxially with respect to the first lumen. The second proximal opening of the tubular guide sleeve extends laterally through the tubular catheter wall. The tubular guide sleeve is connected to the tubular catheter wall in a region of the first distal opening and in a region of the second proximal opening.

Within the meaning of the present invention, the terms proximal and distal are to be considered proceeding from the center of the body of the physician using an aspiration catheter according to the invention.

Within the meaning of the present invention, a lumen is a recess in which there is a complete fluidic communication. An opening in such a lumen can permit fluid contact with a further lumen, for example the interior of a blood vessel or of a suction device.

In a particular embodiment, tubular signifies a substantially round cross section. A cross section is substantially round if it differs by not more than 10% from a circular shape.

In a particular embodiment, the tubular guide sleeve is cohesively bonded to the tubular catheter wall in the region of the second distal opening and in the region of the second proximal opening. That is to say, the two elements, namely the tubular catheter wall and the tubular guide sleeve, are connected in these regions, for example by gluing, welding or by being fused together in such a way that they are connected to each other by molecular forces and are not detachable. Preferably, the distal of the two regions is at the distal end of the tubular catheter wall and extends therefrom a further 1 to 5 mm in the proximal direction, wherein the tubular guide sleeve extends for a further 1 to 10 mm in the distal direction free from the first distal opening, i.e. without contact with the tubular catheter wall.

In a further embodiment, the tubular guide sleeve and the tubular catheter wall are cohesively bonded to each other exclusively in said regions. This means that, over a wide part of an overall longitudinal extent of the aspiration catheter, the guide sleeve extending coaxially with respect to the catheter wall is arranged loosely within the first lumen. This can mean, for example, that only between 0.5 and 8%, in particular between 1 and 5%, of the total longitudinal extent of the guide sleeve are cohesively bonded to the catheter wall.

In a further particular embodiment, the regions in which the tubular catheter wall is cohesively bonded to the tubular guide sleeve each measure between 0.5 and 5 mm, preferably between 1 and 3 mm.

In a particular embodiment, the tubular guide sleeve extends coaxially with respect to the tubular catheter wall over at least part of the longitudinal extent of said tubular catheter wall. The guide sleeve preferably extends over a length of between 20 and 30 cm coaxially with respect to the tubular catheter wall, until it emerges laterally on one side. This part of the aspiration catheter preferably extends from the distal end of the catheter 20 to 30 cm in the proximal direction and, with the second proximal opening of the tubular guide sleeve and the second distal opening of the tubular guide sleeve, forms the rapid exchange port of an aspiration catheter according to the invention. Particularly preferably, the second proximal opening is beveled, that is to say it extends at an angle to the longitudinal axis of the catheter, which is pointed, and preferably measures between 5 and 45°.

In a particular embodiment, the tubular guide sleeve is connected to the catheter wall at its two cohesively bonded connection points in a manner substantially free of stress. Within the meaning of the present invention, the tubular guide sleeve is substantially stress free when, in a straight configuration of the aspiration catheter, no tension exists between the two connection points of the tubular guide sleeve.

In a particular embodiment, the tubular guide sleeve is not cohesively bonded to the tubular catheter wall over a substantial proportion of the part of the longitudinal extent that is coaxial to the tubular catheter wall.

In a particular embodiment, the tubular guide sleeve protrudes from the first lumen at the first distal opening, preferably by a length of between 0.5 and 5 mm. Preferably, the guide sleeve forms, with its second distal opening, the distal end of the whole aspiration catheter. In operation, the second distal opening serves to pierce the thrombus or embolus that is to be aspirated.

In a particular embodiment, the first distal opening is beveled with respect to the diameter of the tubular catheter wall. In a specific example, this means that the first distal opening defines, with a longitudinal axis of the aspiration catheter, an angle of between preferably 35 and 85°, particularly preferably 45 to 70°. The first distal opening is preferably beveled in such a way that the entire opening diameter substantially describes an oval.

In a particular embodiment, the tip of the tubular catheter wall, in particular the distal end of said oval opening, is rounded, such that there is no pointed or sharp edge.

In a preferred embodiment, a distal end of the aspiration catheter is provided with at least one radiopaque marker. The aspiration catheter preferably has a first radiopaque marker on its guide sleeve.

In a particular embodiment, the end of the tubular guide sleeve, in particular the part protruding from the opening, is provided with a radiopaque ring. With the aid of this ring, the position of the distal catheter tip can be observed in the X-ray apparatus and safely monitored in the body.

In a particular embodiment, the aspiration catheter according to the invention has at least one second radiopaque marker. The second radiopaque marker is preferably applied to the aspiration catheter in such a way that the relative position and spatial orientation of the first distal opening can be determined. For this purpose, it can be particularly advantageous for a second or further radiopaque marker to be arranged spaced apart from the first radiopaque marker.

In a particular embodiment, the second or further radiopaque marker is applied to the catheter wall in such a way that a basic orientation of the catheter can be determined. For this purpose, the second or further radiopaque marker has a planar configuration, i.e. the second or further radiopaque marker has an area extent.

The present invention thus relates, in a particular aspect, to an aspiration catheter with a first radiopaque marker at its distal end, preferably on a guide sleeve which protrudes from a first distal opening of a tubular catheter wall, and a second radiopaque marker which is arranged, spaced apart from the first one, on a tubular catheter wall. In a particular embodiment of this aspect, the second or further radiopaque marker is applied as a plaster or patch to the same side of the tubular catheter wall to which the tubular guide sleeve is cohesively bonded. This second or further radiopaque marker is preferably set back, particularly preferably by at least 5 mm, more particularly preferably by between 5 and 45 mm, from the first radiopaque marker.

In a further particular embodiment of this aspect, the second or further radiopaque marker has an area extent which has a long edge and a short edge. In a particular embodiment, this second or further radiopaque marker is rectangular.

In a particular embodiment, the second radiopaque marker is applied to the outside of the tubular catheter wall in such a way that its long edge intersects the longitudinal axis of the aspiration catheter, preferably at a right angle. Particularly preferably, this second or further radiopaque marker extends over a region of the circumference of the tubular catheter wall. Particularly preferably, this region of the circumference of the tubular catheter wall corresponds to the longitudinal extent of the second or further radiopaque marker.

In a particular embodiment, this longitudinal extent describes between 5 and 55, preferably between 10 and 35% of the circumference of the tubular catheter wall.

In a further embodiment of this aspect of the present invention, a second or further radiopaque marker is applied on the opposite side of the catheter wall with respect to the side of the catheter wall to which the tubular guide sleeve is cohesively bonded. Thus, this second or further radiopaque marker is also located on the opposite side from the tip of the tubular catheter wall in a concrete example. In this example too, the second or further radiopaque marker can have a longitudinal extent and a transverse extent.

In a particular embodiment, the longitudinal extent runs in the direction of the catheter longitudinal axis, while the transverse extent runs at a right angle to the catheter longitudinal axis.

In principle, it is known to a person skilled in the art to use radiopaque markers for aspiration catheters. The choice of materials is also known to a person skilled in the art. Radiopaque markers composed of a platinum-iridium (Pt—Ir) compound in the ratio of 90 to 10 are suitable for all aspiration catheters according to the invention.

For example, Pt—Ir rings with an external diameter of 0.6 mm and an internal diameter of 0.5 mm would be suitable for the distal end of the tubular guide sleeve.

Pt—Ir films with a thickness of at least 0.03 mm would be suitable for the second or further radiopaque markers on the tubular catheter wall.

With this structure, the aspiration catheter according to the invention additionally has a sufficient radiopacity to allow it to be reliably located in the body by the physician. This is quite relevant in the case of a total catheter length of approximately 1.5 mm, particularly if improved suctioning of thrombi and/or emboli is intended to be made possible by means of the rotation movement. By means of the abovementioned structural aspect of the present invention, the physician can always detect the orientation of the aspiration opening, even in the case of beveled openings of the aspiration lumina, and can also monitor the effect of the rotation movement.

In a particular embodiment, the total length of the aspiration catheter according to the invention measures 1.5 m.

In a further particular embodiment, the tubular catheter wall is completely or partially coated. The tubular catheter wall preferably has a hydrophilic coating on its outside along its entire length or along part of its length.

In a particular embodiment, the second lumen is designed such that it is suitable for receiving a guidewire.

In a particular embodiment, the aspiration catheter according to the invention is designed such that when the aspiration catheter describes a curve, the tubular guide sleeve always conforms internally to the narrower radius of the curve.

In a particular embodiment, both the tubular catheter wall and the tubular guide sleeve are made of a plastic, particularly preferably a polyamide.

In a particular embodiment, the tubular catheter wall and the tubular guide sleeve are colored differently. The tubular catheter wall is preferably transparent or semi-transparent.

In a preferred embodiment, the aspiration catheter according to the invention has an external diameter of between 1.4 and 2 mm, preferably of between 1.65 and 1.7 mm, particularly preferably of 1.69 mm.

In a preferred embodiment, the tubular guide sleeve has a diameter of between 0.4 and 0.6 mm, preferably of between 0.5 and 0.59 mm, particularly preferably of 0.55 mm.

In a further particular embodiment, the aspiration catheter has a length of approximately 130 cm.

In a particular embodiment, the tubular guide sleeve and the tubular catheter wall of the aspiration catheter according to the invention are made of a PEEK (a polyether ether ketone).

A further aspect of the present invention concerns a component kit for the aspiration of material from blood vessels. Within the meaning of the present invention, a component kit is a series of devices that can be sold as a unit and that can be used together and synergistically for their intended purpose. In the present case, the component kit comprises at least one aspiration catheter. In a particular embodiment, it comprises an aspiration catheter as described above. The component kit further comprises at least one suction device for generating a negative pressure in a first lumen, namely an aspiration lumen, of the aspiration catheter. The component kit further comprises at least one stopcock for connecting the aspiration catheter to the suction device. Within the meaning of the present invention, a stopcock is a device which in principle allows a fluidic connection between two lumina to be converted from a closed state to an open state, and vice versa. In its simplest configuration, the stopcock comprises two openings which can each be connected to a lumen, and a piston which has a bore and can be rotated inside the stopcock in such a way that the bore permits a fluidic connection between the two openings, or the piston blocks this connection. The stopcock is preferably a one-way stopcock.

In combination, a suction arrangement for the aspiration of material from blood vessels would be able to be assembled from distal to proximal with an aspiration catheter, a stopcock and a suction device.

In a particular embodiment, the suction device is a syringe. Suitable syringes are available in the medical trade and have volumes of between, for example, 1 and 50 ml. Conventional manually operated syringes have a piston whose withdrawal from the syringe cavity creates a negative pressure, thereby allowing a suction effect to be generated. Conversely, by pushing the piston into the syringe cavity, the contents of the syringe can be injected through the syringe opening. Alternatively to a manually operated syringe, it is also possible to operate the component kit according to the invention with an automatic suction device. A common aspect of all possible suction devices is that they generate a vacuum and use this in a more or less controlled manner to transmit a negative pressure elements mounted downstream.

In a particular embodiment, the component kit has a coding. Preferably, the component kit has a color coding. By means of such a coding, all the elements are identified with respect to their compatibility.

In routine medical practice, the measure used for the external diameter of catheters is the Charrière unit. The Charrière unit is also given in French (Fr). One French corresponds to a third of a millimeter. It is customary to identify medical catheters on the basis of their external diameter in French.

In a particular embodiment, the component kit is designed for catheters with an order of size of between 6 and 7 Fr. Generally, it is customary to mark 6 Fr with a color coding in orange and 7 Fr with a color coding in green. This makes it easier for medical personnel to identify and use the relevant equipment and the correspondingly correct needles and stylets.

In a particular embodiment, the component kit further comprises a guide catheter for receiving the aspiration catheter in the blood vessels. In practice, after the blood vessels have been punctured, a guide catheter is initially put in place, through which the guidewire is inserted. Later, the aspiration catheter is also pushed along the guidewire through this guide catheter into the vessel.

In a particular embodiment of the present invention, the stopcock has the same coding as the suction device.

The stopcock, in particular the one-way stopcock, is preferably connected via Luer connections to the respective catheter components and/or the suction device. It is crucial that the stopcock is suitable for the corresponding external diameter system. Suitable stopcocks can thus be characterized in the respective colors of the diameter applicable to them.

In a particular embodiment, stopcocks for component kits with an external diameter of 6 Fr are in orange, while stopcocks for a component kit with an external diameter of 7 Fr are in green.

In a further particular embodiment of the component kit according to the invention, the component kit has a further flexible connection element. Preferably, the connection element is designed for connecting the aspiration catheter to the stopcock. Within the meaning of the present invention, flexible is to be understood as meaning that it has an elastic capability. In one particular example, the flexible connection element is made of a plastic selected from the group of plastics having a Shore A hardness of approximately 80. It is particularly preferably a plastic selected from the group consisting of polyurethanes, silicones and PVC.

In operation, the flexible connection element is connected to the proximal end of the aspiration catheter. The flexible connection element is then connected with a stopcock to a suction device. In operation, a vacuum is firstly generated between the suction device and the stopcock. Thereafter, the aspiration catheter is introduced via the guide catheter into a punctured blood vessel. The flexible connection element serves as a hinge and facilitates the insertion of the catheter.

A further particular aspect of the present invention concerns a method for the aspiration of material from blood vessels. The method comprises initially making available an aspiration catheter, in particular an aspiration catheter according to the invention as described in the introduction. By means of a stylet or a needle, the blood vessel is punctured to permit insertion of a guidewire into the blood vessel. A guidewire is then placed in the blood vessel. The aspiration catheter is now inserted with the aid of the guidewire, and the material to be suctioned, for example the thrombus or embolus to be aspirated, is pierced with the tip of the aspiration catheter. The aspiration catheter is then withdrawn, with execution of a rotation movement about the longitudinal axis of the aspiration catheter, and aspiration is simultaneously performed by application of a vacuum.

In the preparation of the catheter, it is also possible, in a particular embodiment, for a guide catheter also to be first introduced into the blood vessel. This guide catheter facilitates the insertion of the guidewire and any future changes of catheter.

During the use of an aspiration catheter according to the invention, the flapping and twisting movement of the catheter during rotation about its own longitudinal axis is reduced. This on the one hand permits more accurate aspiration and on the other hand avoids uncontrolled flapping movement and any suctioning of the inner wall of the vessel.

For a person skilled in the art, it is obvious that all of the embodiments that have been discussed above can, when not mutually exclusive, be implemented in any desired combination in a configuration of the aspiration catheter according to the invention or the component kit.

The aspiration catheter according to the invention is in principle suitable for veins and/or arteries.

However, uses in other vessels and cavities of the body are also entirely conceivable and can be inferred by a person skilled in the art from the disclosure of the invention.

The invention is explained in more detail below with reference to figures and to specific illustrative embodiments, but without limiting the scope of protection thereto.

### BRIEF DESCRIPTION OF THE DRAWINGS

In the drawings used to explain the illustrative embodiments:

FIG. 1 shows schematically the distal end, with the rapid exchange port, of an aspiration catheter according to the invention;

FIG. 2 shows schematically an alternative embodiment with an inserted guidewire;

FIG. 3 shows schematically the distal end of an aspiration catheter according to the invention with radiopaque markers;

FIG. 4 shows schematically an aspiration catheter according to the invention in an external view;

FIG. 4a shows the catheter tip schematically in a plan view;

FIG. 4b shows a cross section in the section plane A-A;

FIG. 4c shows a cross section in the section plane A′-A″;

FIG. 4d shows a cross section in the section plane A″-A″;

FIG. 5 shows a cross section through an aspiration catheter from the prior art;

FIG. 6 shows schematically a distal end of an aspiration catheter according to the invention with alternative radiopaque markers;

FIG. 7 shows schematically a component kit according to the invention, and

FIG. 8 shows schematically the placement of an aspiration catheter according to the invention.

In principle, analogous elements are provided with the same reference signs in the figures.

### WAYS OF IMPLEMENTING THE INVENTION

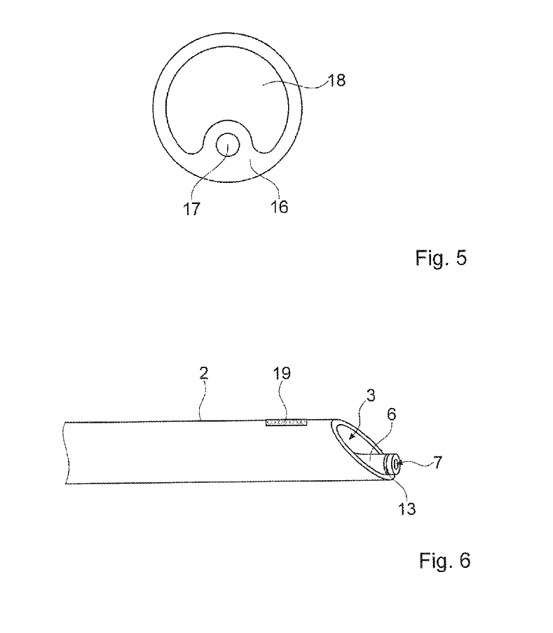

FIG. 5 shows an example of a cross section through a common aspiration catheter of the kind known and used in the prior art. The catheter wall 16 is designed such that it encloses an aspiration lumen 18 and a guidewire lumen 17. It is clearly evident that if such a cross section extends through the entire length of the region guided by the guidewire, there is unilateral stiffness on the side of the catheter wall at which the guidewire lumen 17 is formed. When such a catheter is rotated about its own axis, it thus performs a twisting or flapping movement.

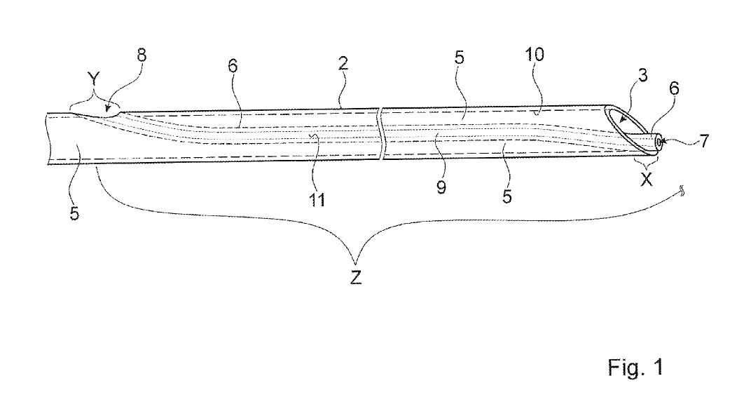

FIG. 1 shows schematically an embodiment of the aspiration catheter according to the invention. The depicted region Z corresponds more or less to the guided region, or the rapid exchange port Z. The aspiration catheter has a tubular catheter wall 2 which defines a first lumen 5 in its interior. The external diameter of the catheter wall 2 in this specific example is 1.69 mm. The wall thickness is approximately 0.075 mm. The catheter inner wall 10 encloses the first lumen 5 which, at its distal end, can be brought into fluidic communication with a vessel interior (not shown) via a first distal opening 3. The proximal end of the tubular catheter wall 2 is not shown in this example. A tubular guide sleeve 6 with an external diameter of 0.55 mm extends inside of the tubular catheter wall 2. This guide sleeve 6 in turn defines, via a guide sleeve inner wall 11, a second lumen 9 which serves as a guidewire lumen 9. At the distal end, the guide sleeve 6 protrudes by between 0.5 and 8 mm from the tubular catheter wall 2 and has a second distal opening 7.

The proximal end of the tubular guide sleeve 6 extends laterally through the tubular catheter wall and there forms a second proximal opening 8. In this drawing, the opening is shown purely schematically on the opposite side from the tip of the guide sleeve. The lateral opening can emerge at any desired point of the circumference of the tubular catheter wall 2.

The tubular guide sleeve is connected to the tubular catheter wall 2 exclusively in the regions X, Y, that is to say in a region of the first distal opening and in a region of the second proximal opening. In the present example, both the tubular catheter wall 2 and the tubular guide sleeve 6 are made of PEEK and are cohesively bonded to each other, for example welded to each other, in the regions X, Y. Preferably, there is almost no stress, or only negligible stress, at the weld points in the regions X, Y, when the catheter is straight and stretched.

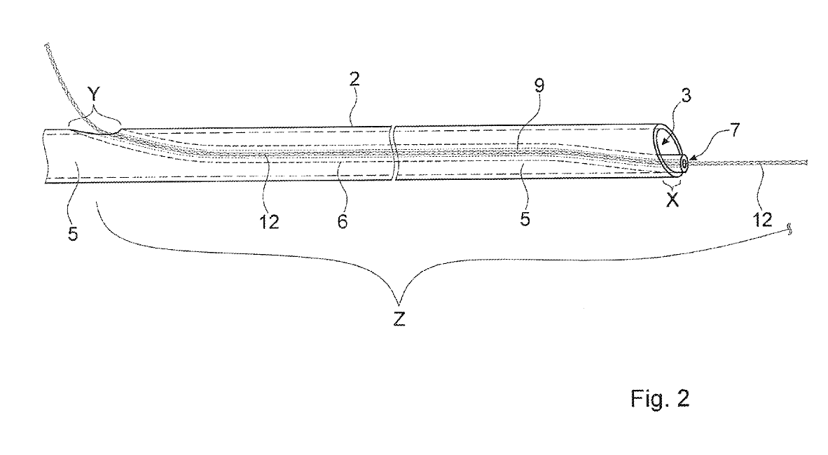

In FIG. 2, the depicted rapid exchange port Z is shown with an inserted guidewire 12, which extends through the entire longitudinal axis of the tubular guide sleeve 6. In operation, the guide wire 12 is inserted into the second distal opening 7 and guided back out again through the second proximal opening 8. With the aid of this guide, the catheter is advanced through the blood vessel as far as the obstruction. The catheter wall 2 in this illustrative embodiment has a first distal opening 3 that has a less acute angle than in FIG. 1. In the present example, the tip of the aspiration catheter has an angle of 45°. In this illustrative embodiment too, the tubular guide sleeve is cohesively bonded to the tubular catheter wall 2 at its lateral exit point Y and at a contact point X at the distal opening 3. The tubular guide sleeve thus extends through the lumen 5 of the tubular catheter wall 2.

Although the radiopaque markers are not shown in the two illustrative embodiments depicted in FIGS. 1 and 2, they may of course be provided at the distal end of the aspiration catheter.

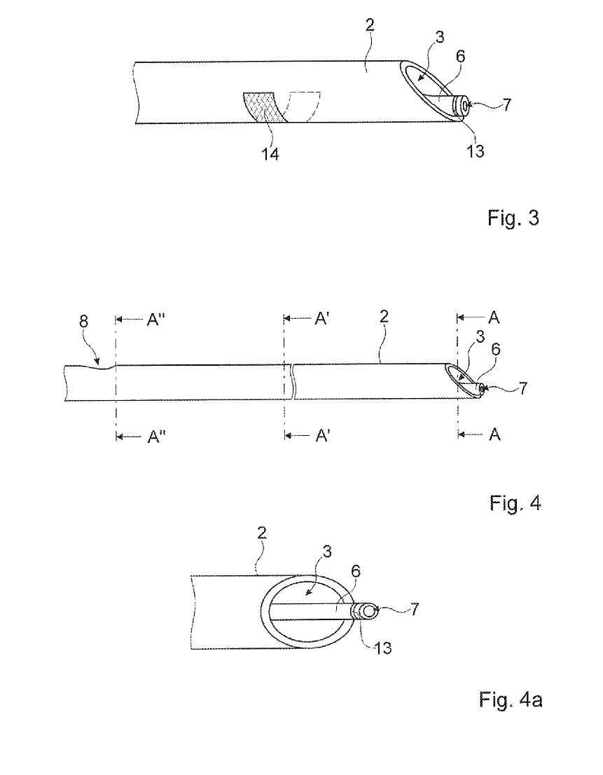

FIG. 3 shows schematically an illustrative embodiment with suitably placed radiopaque markers 13, 14. It shows the distal end of an aspiration catheter according to the invention. The tubular catheter wall 2 terminates with a beveled aspiration opening 3, through which the tubular guide sleeve 6 extends with its distal opening 7. The tubular guide sleeve 6 is marked with a radiopaque PT—IR ring at its distal end. In the proximal direction, spaced apart from this first radiopaque marker 13, a radiopaque Pt—Ir film 14 is applied externally on the catheter wall 2 on the same side as the adhesive point of the guide sleeve. This marker can be glued on, welded on or soldered on and has a material thickness of 0.03 mm. In the example shown, the film 14 is designed such that it surrounds the circumference of the tubular catheter wall 2 at least by half. By the geometry of this radiopaque film, the orientation of the aspiration catheter can be visualized on the X-ray apparatus. On the basis of the opening of the film, an operating surgeon can determine in which direction the oval aspiration opening 3 opens.

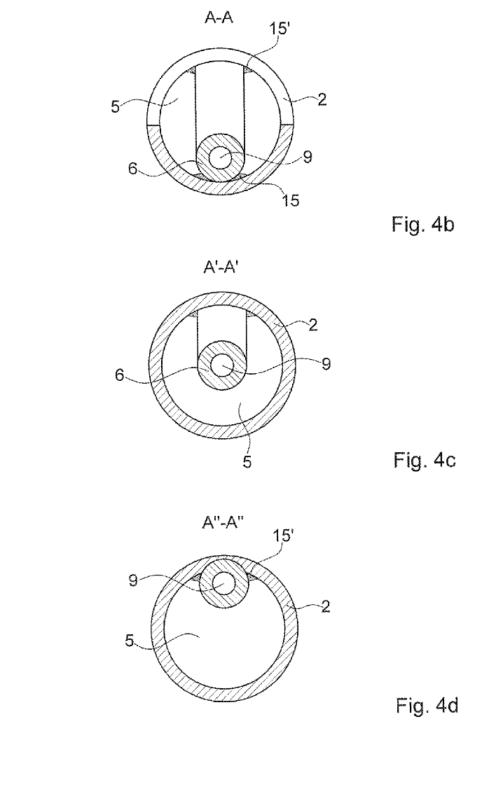

For better illustration, FIG. 4 again shows the catheter tip according to the invention with the catheter wall 2, the first distal opening 3, the second distal opening 7, the tubular guide sleeve 6 and the second proximal opening 8 in various cross sections.

FIG. 4a shows the oval first distal opening 3, having the tubular guide sleeve 6 extending therefrom with the second distal opening 7 and the radiopaque Pt—Ir ring 13.

FIG. 4b illustrates the cross section in the section plane A-A of FIG. 4 at the catheter tip. The tubular guide sleeve 6 is cohesively bonded to the catheter wall 2 by means of a weld seam 15 and extends rearward from the section plane through the aspiration lumen 5 as far as its outlet, where it is once again cohesively bonded to the catheter wall via a weld seam 15′.

FIG. 4c illustrates the intermediate space between the two openings of the guide sleeve in the section plane A′-A′. The guide sleeve 6 is arranged freely in the first lumen 5 of the tubular catheter wall 2.

FIG. 4d shows the proximal end of the guide sleeve, where the guide sleeve 6 is once again cohesively bonded to the tubular catheter wall 2 via a weld seam 15′.

FIG. 6 shows an alternative or supplementary arrangement of the radiopaque markers 13, 19. Besides the broad connection to the radiopaque ring 13 discussed in FIG. 3 at the distal end of the tubular guide sleeve, this aspiration catheter has a further rectangular Pt—Ir film 19 which on the opposite side, specifically a radiopaque film 19 applied with a longitudinal edge in the longitudinal direction of the aspiration catheter. With these markers, it is likewise possible for a surgeon to reliably determine the position and orientation of the aspiration catheter.

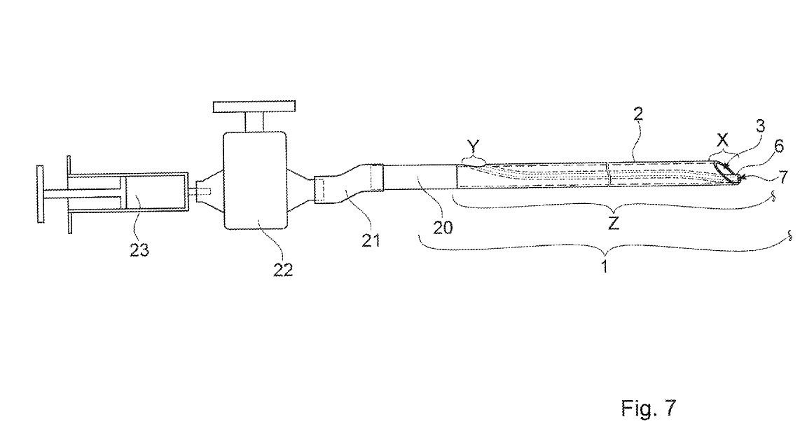

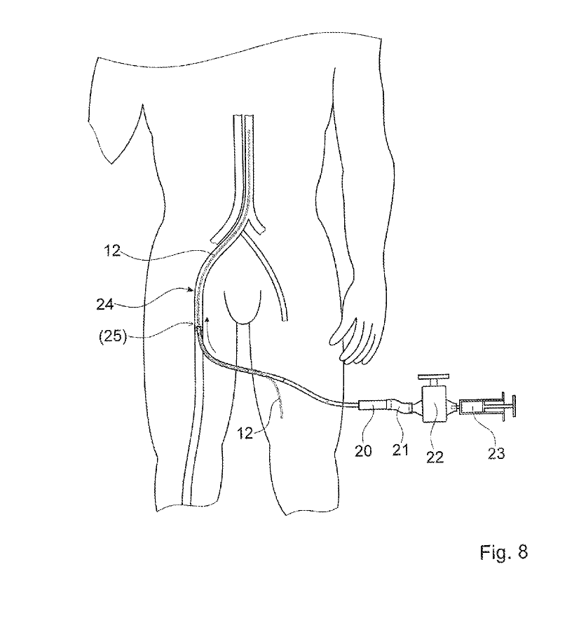

In FIG. 7, a component kit according to the invention is shown schematically and not to size. The aspiration catheter 1 according to the invention comprises a distal rapid exchange port Z which, in addition to a tubular catheter wall 2, comprises a tubular guide sleeve 6 that is freely movable within the aspiration lumen 5, except for its regions Y, X. This guide sleeve 6 is cohesively bonded to the tubular catheter wall 2 via its contact regions X, Y. The rapid exchange port Z is approximately 20 to 30 cm long and ends in the catheter part 20, which is a further 80 to 100 cm long. The rapid exchange port Z can be cohesively bonded to this catheter part 20. However, it is also conceivable that the tubular catheter wall 2 is integrally formed with the catheter part 20 and represents a continuation of the tubular catheter wall. Overall, the entire length of the catheter part 20 forms, with the rapid exchange port Z, the aspiration catheter 1 which, at its proximal end, is connected by a flexible PU hose 21 to a one-way stopcock 22. The latter is in turn connected to a syringe 23.

FIG. 8 shows an example of the use of a component kit according to the invention with a corresponding aspiration catheter. Such a method would be used, for example, in percutaneous thrombus aspiration, in which the femoral artery (Arteria femoralis) is punctured and a guidewire is guided into the artery, through the aorta and into the heart.

A guide catheter is used for placement of the guide wire. This guide catheter can belong, for example, to a 6 Fr or 7 Fr catheter system. The choice of the guide catheter determines the selection of the corresponding subsequent components, wherein stopcock 22 and syringe 23 and the aspiration catheter to be used can likewise have a corresponding color coding.

When the guide catheter is removed again, the aspiration catheter can be guided via the puncture 25 through the femoral artery 24 to the obstruction site (not shown). The obstruction site is pierced by the aspiration catheter and then, while the aspiration catheter is being withdrawn, aspiration is carried out.

At the same time, a rotation movement of the aspiration catheter is effected, by which the aspiration takes place as uniformly as possible. In principle, a negative pressure is generated by means of the syringe even before insertion of the aspiration catheter, with the stopcock closed. When the stopcock is opened, the negative pressure creates a suction effect through the lumen of the catheter as far as the distal opening of the aspiration catheter. At the start of the procedure, it may be advisable to flush the catheter or the syringe with physiological saline solution.

The aspiration catheter according to the invention and the component kit can of course also be used for thrombi and emboli at other locations of the body.

1-15. (canceled)

16. An aspiration catheter for the aspiration of material from blood vessels, comprising
a tubular catheter wall having a first distal opening and a first proximal opening, which are connected by a first lumen;
a tubular guide sleeve having a second distal opening and a second proximal opening, which are connected by a second lumen, and wherein
the tubular guide sleeve is arranged inside the first lumen, and wherein
the second lumen extends coaxially with respect to the first lumen, and wherein
the second proximal opening extends laterally through the tubular catheter wall, and
wherein the tubular guide sleeve is connected to the tubular catheter wall in a region of the first distal opening and in a region of the second proximal opening.

17. The aspiration catheter as claimed in claim 16, wherein the tubular guide sleeve is cohesively bonded to the tubular catheter wall in the region of the first distal opening and in the region of the second proximal opening,

18. The aspiration catheter as claimed in claim 17 wherein the tubular guide sleeve is cohesively bonded to the tubular catheter wall exclusively in said regions.

19. The aspiration catheter as claimed in claim 16, wherein the tubular guide sleeve extends coaxially with respect to the tubular catheter wall along at least a part of the longitudinal extent of said tubular catheter wall.

20. The aspiration catheter as claimed in claim 19, wherein the tubular guide sleeve is not cohesively bonded to the tubular catheter wall along 75% to 99% of the length of this part.

21. The aspiration catheter as claimed in claim 16, wherein the tubular guide sleeve protrudes from the first lumen at the first distal opening.

22. The aspiration catheter as claimed in claim 16, wherein the second distal opening forms the distal end of the aspiration catheter.

23. The aspiration catheter as claimed in claim 16, wherein the first distal opening is beveled with respect to the diameter of the tubular catheter wall.

24. The aspiration catheter as claimed in claim 16, wherein the aspiration catheter has at least one first radiopaque marker at its distal end.

25. The aspiration catheter as claimed in claim 24, wherein the aspiration catheter has at least one further radiopaque marker.

26. The aspiration catheter as claimed in claim 25, wherein the at least one further radiopaque marker is spaced apart from the first radiopaque marker.

27. The aspiration catheter as claimed in claim 16, wherein the aspiration catheter comprises at least one first radiopaque marker and at least one second radiopaque marker, and wherein the second radiopaque marker has an area extent by which the orientation of the first distal opening can be determined on the basis of an X-ray image.

28. A component kit for the aspiration of material from blood vessels, comprising the following elements:
at least one aspiration catheter as claimed in claim 16;
at least one suction device for generating a negative pressure in the first lumen of the aspiration catheter;
at least one stopcock for connecting the aspiration catheter to the suction device.

29. The component kit as claimed in claim 28, wherein the component kit has a coding which identifies all of said elements according to their external diameter and their compatibility.

30. The component kit as claimed in claim 29, wherein the coding is a color coding.

31. The component kit as claimed in claim 29, wherein the stopcock has the same coding as the suction device.

32. The component kit as claimed in claim 31, wherein the coding of the stopcock is a coding according to its Charrière diameter.

33. The component kit as claimed in claim 28, wherein the component kit further comprises a flexible connection element for connecting the aspiration catheter to the stopcock.

34. The component kit as claimed in claim 33, wherein the flexible connection element is made of a material that has a Shore A hardness of approximately 80.

35. A method for the aspiration of thrombi or emboli from blood vessels, said method comprising the steps of:
making available an aspiration catheter as claimed in claim 16;
puncturing a blood vessel in order to insert a guidewire into the blood vessel;
positioning the guidewire in the blood vessel;
inserting the aspiration catheter with the aid of the guidewire and piercing the thrombus or embolus that is to be aspirated;
withdrawing the aspiration catheter while at the same time aspirating the obstructing material.
